(19) United States
(12) Patent Application Publication
(10) Pub. No.: US 2005/0177232 A1
(43) Pub. Date: Aug. 11, 2005

(54) Implantable organ encircling band

(76) Inventor: Sally Ann Ashton, Bristol (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/504,801

(22) PCT Filed: Feb. 12, 2003
(86) PCT No.: PCT/GB03/00649

(30) Foreign Application Priority Data
Feb. 13, 2002 (GB) 0203332.2

(51) Int. Cl. A61F 2/16

(52) U.S. Cl. 623/6.63; 606/151

(57) ABSTRACT

A medical device for encircling and constricting an organ is described herein. The device consists of a linear planar band of a surgically-acceptable material which has an integral fastener of mutually interlocking parts at the respective ends of the band allowing the ends of the band to be securely fastened to one another. The device is preferably used as an implantable retinal encirclement band.

This invention relates to a medical device, more particularly the present invention relates to a device for correcting or reconfiguring a deformed region or organ of the body.

The present invention will be described with reference to its preferred embodiment as a correction device for retinal detachment. However, the present invention finds equal utility in other areas of the body where it is desired that a hollow or deformable organ or region be reconfigured, for example, the constriction of a blood vessel or other conduit, or the inflation or re-opening of a prolapsed vessel or organ.

Retinal detachment is an ophthalmic condition where the epithelial and neural layers of the retina separate. The condition can be caused by injury, short-sightedness, diabetes or separation of the vitreous from the retina, although there are other causes. This condition, if not detected and treated early, generally results in partial or total blindness. Retinal detachment may be diagnosed as a result of the visual perception of “sparks” of light, “lightning flashes” or “floaters” or a shadow in the field of vision by the patient. In a healthy individual, the retina is opposed to the underlying retinal pigment epithelial layer.

There are several methods employed by the medical profession to reattach a detached retina, irrespective of the cause. Such methods include cryotherapy, laser treatment, plombage or encirclement. Encirclement methods are generally used when there is a large area of the retina to be treated or when many splits, holes or small detachments are present.

Generally, encirclement methods use a retinal encircling band to apply pressure to the eye deforming it and ensuring that the detached portion of the retina is supported.

For example, RU0888990 describes an implant for scleroplastic operations; the implant is in the form of a splint encased in a sleeve attached to a textile band which can be stitched to the eye. In use, the band is stitched to the eye and the ends of the splint drawn together through the sleeve until the desired level of deformation of the eye is achieved. This device is difficult to use and the stitching of the textile band to the eye is time-consuming for the surgeon.

U.S. Pat. No. 6,117,170 describes a sclera band for supporting the choroid beneath the retina during a so-called “welding” operation to reattach the retina to the choroid such as thermocoagulation or cryotherapy. The band includes a region which is differently compressible to the remainder of the band and which forms the supporting region. In use, the supporting region is positioned below the area where the retina is detached and used to hold the underlying support area in position below the retina for reattachment by further surgical intervention.

Additionally, a plain band with a buckle or Waski sleeve may be used, the band being encircled about the eye and the ends of the band drawn together and pulled through a buckle or Waski sleeve. This is time consuming and fiddly for the surgeon and often necessitates the use of an assistant surgeon to assist with measuring the circumference of the eye, cutting the sleeve to size and placing the ends of the band through the buckle or Waski sleeve to secure the band about the eye. Additionally, if the ends are not securely fastened, there is a risk that the band will become slack due to relative sliding of the ends of the band.

It is therefore an object of the present invention to provide a device which is less time-consuming to use and is simple to fasten.

Accordingly, the present invention provides an organ-encircling device, the device consisting of a planar band of a surgically-acceptable material with an integral fastener having a plurality of mutually interlocking parts at the respective ends thereof whereby, in use, the ends of the band are passed about the organ and the mutually interlocking parts are introduced to one another and fastened.

Advantageously, a band of this type is easier for a surgeon to fit since there is no threading or pulling of the respective ends of the band through a sleeve or buckle and hence there is a reduced risk of the band becoming slack in use by virtue of relative sliding of the ends of the band.

In the most preferred embodiment, the present invention provides an implantable retinal encirclement band consisting of a planar band of a surgically-acceptable material with an integral fastener having a plurality of male and female mating parts at the respective ends thereof whereby, in use, the ends of the band are passed about the eyeball and at least one pair of the male and female mating parts are introduced to one another and fastened.

Optionally, the band is releasable.

Preferably, the band is linear or rectilinear along its length, The term “linear” as used herein is intended to describe a band which is not curved or arcuate in its pre-use configuration but which can be moved into a curved or arcuate configuration, for example by folding, when used to encircle an organ, by virtue of the properties of the material-from which it is made.

Preferably, the fastener comprises pairs of mutually interlocking parts. More preferably the fastener comprises no more than six pairs of mutually interlocking parts since this would render the band unacceptably bulky. However, the band may have more than six pairs of pairs of mutually interlocking parts where the organ to be encircled is large or not rigid. Optimally, for use as a retinal encirclement band, the fastener comprises two to four pairs of mutually interlocking parts, and ideally three pairs of mutually interlocking parts, since this provides sufficient adjustability to the device without being unacceptably bulky.

The mutually interlocking parts of the fastener are preferably of the snap-fit type to allow for simple fastening of the device, however other forms of interlocking fastener may be used with equal utility.

In the preferred embodiment the fastener has interlocking parts in the form of projecting members such as upstanding lugs, pegs or the like at one end of the band and notches, holes, slots, cups or the like receiving members at the other. Any holes provided may be blind. Effectively, any reciprocating male and female type mating parts may be used. The band may be locked by introducing more than one lug or peg into more than one respective slots or holes, for example two lugs may be introduced into two holes. Where slots or holes are used, they may be profiled to impede release of the lug. For example, a hole may be dimensioned such that the lug is force fitted into the hole by causing deformation of the hole. The or each hole may be provided with a region of reinforcement to prevent the hole from distending, splitting or tearing. Such a reinforced region may extend beyond the planar surface of the band in either direction.

Alternatively, the hole or slot may be shaped so that a lug may be introduced to the hole or-slot and then moved into a position where the lug or hole is smaller to prevent accidental release of the lug but to allow deliberate release of the fastener. For example, a keyhole shaped slot may be used where, in use, the lug is introduced to the slot in the wider region and subsequently moved into and held in the narrower region. In such an embodiment the lug may be provided with a notch which interacts with the hole to impede accidental release of the lug from the hole.

The lugs, pegs or the like may themselves be shaped or profiled to impede their subsequent release. For example, the end of the lug distal to the band may flare outwardly to provide a region which is larger than the main body or shaft of the lug. The use of a flared region facilitates force-fitting of the fastener. In another embodiment, the lug or the like may be provided in one or more sections each having an enlarged region at the distal end, and being capable of deforming toward one another as they are passed through the hole and moving apart again when the enlarged region has been passed through the hole.

However, the lug or the like may be moulded to provide an enlarged region at the distal end without flaring or tapering for force fitting by deformation of the hole, or for fitting in the keyhole-type slot described above.

The interlocking parts of the fastener may be arranged such that only lugs or pegs are located at one end of the band and only slots or holes at the other, or there may be a mixture of lugs or pegs and slots or holes at each end of the band.

The band may have an unequal number of fastener parts at its respective ends, for example there may be three lugs and five slots or holes for receiving the lugs. Alternatively, there may be four lugs and a single hole for attachment to any one of the lugs, or there may be four holes one of which engages a single lug.

Alternatively, a series of holes or like receiving members may extend from the region of the upstanding lugs or pegs to the opposite end of the band or vice versa. Provision of such an array of holes would allow adjustment over a wide range of sizes of organ.

In a preferred embodiment, in use, the respective ends of the band overlie one another lengthwise. Preferably, the band is straight and the longitudinal edges of the overlying ends are flush with one another.

The device of the present invention may also be provided with a reinforced region along the edges of the band. This region may include some profiling, such as notches or indentations which allow the insertion of forceps by the user for example, in order to hold the band while bringing the opposite end of the band about the eye or other organ.

The band of the present invention is intended to remain in situ in the patient that is, the band can be implanted. In some cases it may be desirable for the band to can be removed during a further surgical procedure, once it had been established that the retina has sufficiently reattached to allow the removal of the band. Optionally, however, the band of the present invention may be biodegradable to prevent further surgery being necessary to remove the band. In any event, it is preferred that the band is left in place at least until reattachment has occurred. Where the band is biodegradable, it is preferred that the degradation is slow, optimally over a period in excess of one year to allow reattachment to occur. The band may be made of any suitable biodegradable polymer.

The band of the present invention may be coated or impregnated with pharmaceutically acceptable materials such as antibiotics, anti-inflammatory agents, or agents to promote or impede adhesion of the band to the choroid.

The term “surgically acceptable material” as used herein is intended to describe any synthetic material such as a polymer, an elastomeric material or plastics material which has been shown to have non-allergenic effects, be capable of being sterilised and be sufficiently robust to be used in standard surgical procedures. The surgically acceptable material presently preferred for the band of the invention is surgical grade silicone rubber. The material selected to produce the band of the present invention generally has equal compressibility throughout its length. The band may be resilient to allow some tension to be applied to the encircled organ or to allow for some expansion of the organ, under tension, during use.

Additionally, the band may comprise indicia means giving an indication of the diameter of the loop or circle defined by the band. The indicia means may be printed or embossed. Advantageously, if the surgeon had previously determined the diameter of the eyeball, or other organ to be encircled, then they could cut the band to size prior to surgery and simply align the relevant fasteners at the indicia mark and fasten the band, thereby speeding up the surgical procedure.

The band of the present invention may be formed by any known method for producing shaped polymers, plastics and the like although it is preferred that the band is produced by moulding, especially injection or vacuum moulding.

In use, the band is used to encircle an eye by wrapping it about an eye in the correct orientation for treatment. The surgeon or other medical practitioner then aligns the respective mating parts of the fastener with one another when the length of the band is such that it will apply the correct amount of local pressure to the eye; this amount having been determined by the surgeon or other medical practitioner. Force is applied to the fastener from one or both sides, for example using a pair of forceps, in order to engage the mutually interlocking parts and thereby fasten the fastener.

Embodiments of the present invention will now be described by way of example only, with reference to the appended drawings, of which

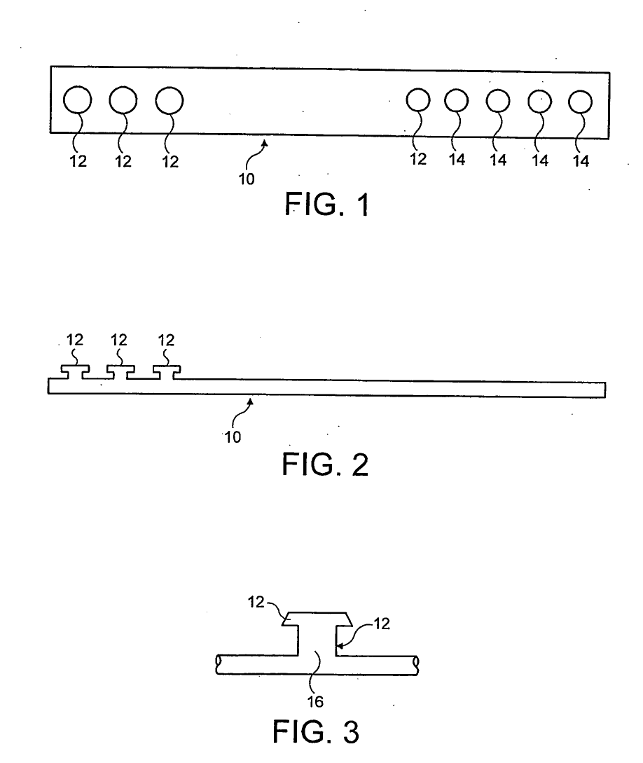

FIG. 1 is a plan view of the retinal band of the present invention from above;

FIG. 2 is a side-elevation of the band of FIG. 1, and

FIG. 3 shows a detail of the lugs of FIGS. 1 and 2.

Referring to FIGS. 1 and 2, a retinal encirclement band 10 is provided in accordance with the present invention which comprises three lugs 12 and five holes 14. The lugs 12 are provided as an array at one end of the band 10 while the holes 14 are provided as an array at the other end of the band i 0.

As can be seen more clearly in FIG. 3, each lug 12 has a region 13 of larger diameter than the shaft 16 of the lug 12.

In use, the band 10 is wrapped about the eye, generally at or near the equator thereof, and the ends are introduced to one another. The surgeon overlays the end bearing holes 14 over the end bearing lugs 12 until the desired diameter of the band is reached. The lugs 12 are then forced through the overlying holes 14 to securely fasten the ends together.

1. An organ-encircling device, the device consisting of a planar band of a surgically-acceptable material with an integral fastener having a plurality of mutually interlocking parts at the respective ends thereof whereby, in use, the ends of the band are passed about the organ and the mutually interlocking parts are introduced to one another and fastened.

2. A device according to claim 1, in which the band is linear or rectilinear.

3. A device according to claim 1 or claim 2, in which the fastener comprises pairs of mutually interlocking parts.

4. A device according to claim 3, in which the fastener comprises less than six pairs of mutually interlocking parts.

5. A device according to claim 3 or claim 4, in which the fastener comprises three pairs of mutually interlocking parts.

6. A device according to any preceding claim, in which the mutually interlocking parts of the fastener are of the snap-fit type.

7. A device according to any preceding claim, in which the fastener comprises reciprocating male and female type mating parts.

8. A device according to any preceding claim, in which the fastener has interlocking parts in the form of complementary upstanding members and receiving members.

9. A device according to claim 8, in which the upstanding members are lugs, and are provided at one end of the band and the receiving members are holes and are provided at the other.

10. A device according to claim 8, in which a mixture of lugs and holes are provided at each end of the band.

11. A device according to claim 9 or claim 10, in which an unequal number of lugs and holes are provided at the respective ends of the band.

12. A device according to claim 9, in which the holes are provided along the length of the band from the lugs to the opposite end of the band.

13. A device according to any one of claims 9 to 12, in which the lugs, are shaped or profiled.

14. A device according to claim 13, in which the lug is provided with an enlarged region its distal end.

15. A device according to claim 13, in which the end of the lug distal to the band is flared to provide a region which is larger than the shaft of the lug.

16. A device according to any one of the preceding claims in which the surgically acceptable material is suitable for implantation.

17. A device according to any one of the preceding claims, in which the surgically-acceptable material is surgical-grade silicone rubber.

18. A device according to any one of the preceding claims, in which the surgically-acceptable material is biodegradable.

19. A device according to claim 18, in which the surgically-acceptable material biodegrades after 12 months.

20. A device according to any one of the preceding claims, in which the surgically-acceptable material is coated or impregnated with a pharmaceutically acceptable compound.

21. A device according to claim 20, in which the pharmaceutically acceptable compound is an antibiotic, an anti-inflammatory or a promoter or inhibitor of adhesion.

22. A device according any one of the preceding claims, in which the band is provided with indicia means indicating the length of the band.

23. A device according to claim 22, in which the indicia means are printed or embossed.

24. A device according any one of the preceding claims for use in ophthalmic surgery.

25. Use of a device according any one of the preceding claims as a retinal encirclement band.

26. An implantable retinal encirclement band consisting of a linear planar band of a surgically-acceptable material with an integral fastener having a plurality of male and female mating parts at the respective ends thereof whereby, in use, the ends of the band are passed about the eyeball and at least one pair of the male and female mating parts are introduced to one another and fastened.

27. A method of treating a detached retina, the method comprising the steps of encircling an eyeball with an implantable retinal encirclement band consisting of a linear planar band of a surgically-acceptable material with an integral fastener having a plurality of male and female mating parts at the respective ends thereof and fastening at least one pair of the male and female mating parts.

28. A device, substantially as described herein and as illustrated by FIGS. 1 to 3 of the accompanying drawings.
